(19) United States
(12) Patent Application Publication
(10) Pub. No.: US 2015/0282953 A1
(43) Pub. Date: Oct. 8, 2015

(54) COMPRESSION HEEL PROSTHETIC FOOT

(71) Applicant: Ability Dynamics, LLC, Tempe, AZ (US)

(72) Inventors: Keith B. Smith, Gilbert, AZ (US); Gene Parker, Mesa, AZ (US)

(21) Appl. No.: 14/731,818

(22) Filed: Jun. 5, 2015

Related U.S. Application Data
(63) Continuation of application No. 13/568,535, filed on Aug. 7, 2012.
Application No. 13/568,535 is a continuation of application No. 13/568,535, filed on Aug. 7, 2012.
Application No. 13/568,535 is a continuation-in-part of application No. PCT/US11/33319, filed on Apr. 20, 2011.
Application No. PCT/US11/33319 is a continuation-in-part of application No. 12/799,215, filed on Apr. 20, 2010, now abandoned.
Application No. 12/799,215 is a continuation-in-part of application No. 11/901,845, filed on Sep. 19, 2007, now Pat. No. 8,048,173.
Application No. 11/901,845 is a continuation-in-part of application No. 13/642,501, filed on Nov. 27, 2012, now Pat. No. 9,078,773.
Application No. 13/642,501 is a continuation-in-part of application No. 12/799,215, filed on Apr. 20, 2010, now abandoned.
Application No. 12/799,215 is a continuation-in-part of application No. 11/901,845, filed on Sep. 19, 2007, now Pat. No. 8,048,173.

(51) Int. Cl. A61F 2/66

(52) U.S. Cl. CPC A61F 2/66; A61F 2002/6614; A61F 2002/6642

(57) ABSTRACT

A prosthetic foot comprises a resilient bottom member, a resilient top member, wherein the anterior top end is connected to the anterior bottom end of the resilient bottom member, and wherein the resilient top member is positioned over the resilient bottom member, and an elastomeric bumper member comprising a tapered surface configured to contact the resilient bottom member and attached to an underside of the posterior of the resilient top member. The prosthetic foot can further comprise a toe pad having a spacer coupled to, and creating space between, the bottom member and the top member, and an adhesive bonding one of the bottom member and one end of the top member, where the adhesive is commingled with the spacer between the first bottom end and the first top end.

### CROSS-REFERENCES TO RELATED APPLICATIONS

This application is a continuation of U.S. patent application Ser. No. 13/568,535, filed on Aug. 7, 2012; and this application is a continuation of U.S. patent application Ser. No. 13/568,535, filed on Aug. 7, 2012, which is a continuation-in-part of International Application No. PCT/US11/33319, filed on Apr. 20, 2011, which is a continuation-in-part of U.S. patent application Ser. No. 12/799,215, filed on Apr. 20, 2010, which is a continuation-in-part of U.S. patent application Ser. No. 11/901,845, filed on Sep. 19, 2007, now U.S. Pat. No. 8,048,173; and this application is a continuation-in-part of U.S. patent application Ser. No. 13/642,501, filed on Nov. 27, 2012, which is a 371 national phase application of International Application No. PCT/US11/33319, filed on Apr. 20, 2011, which is a continuation-in-part of U.S. patent application Ser. No. 12/799,215, filed on Apr. 20, 2010, which is a continuation-in-part of U.S. patent application Ser. No. 11/901,845, filed on Sep. 19, 2007, now U.S. Pat. No. 8,048,173 and incorporates the disclosure of all such applications by reference, and this application incorporates the disclosure of all such applications by reference.

### FIELD OF THE INVENTION

This invention pertains to prosthetic devices. More particularly, the invention pertains to a prosthetic foot that, when utilized by an amputee, better replicates the action of a real foot and reduces the risk of injury to the amputee.

### BACKGROUND OF THE INVENTION

Prosthetic feet are well known in the art. In use, such prosthetic feet typically do not replicate the action of a real foot and can generate “kickback” or “kickforward” reactions that increase the risk of injury to an amputee utilizing the foot. Kickback is motion created by the prosthetic foot in a backward direction during the walking cycle. Kickforward is motion created by the prosthetic foot in a forward direction during the waling cycle. Either motion may create instability for user if expanding or restricting the intended motion. Further, many prior art prosthetic foot generate vibrations that can travel through a user's leg and cause discomfort.

For an amputee, loosing bipedality may produce an involuntary anterior lean or shift, forcing a constant imbalance or rebalance of posture. The amputee no longer possesses voluntary muscle control on his involved side due to the severance of the primary flexor and extensor muscles. The primary anterior muscle responsible for dorsiflexion (sagittal plane motion) is the anterior tibialis. Dorsiflexion is the voluntary ankle motion that elevates the foot upwards, or towards the midline of the body. The primary posterior muscle responsible for plantarflexion is is the gastro-soleus complex. It is a combination of two muscles working in conjunction: the gastrocnemius and the soleus. Plantarflexion is the voluntary ankle motion that depresses the foot downwards, or away from the midline of the body. Therefore, it is desirable to have a prosthetic foot configured to promote increased muscle activity and promote increased stability for amputees, and it is desirable to provide an improved prosthetic foot which would better replicate the action of a true foot. Furthermore, it is desirable to provide an improved prosthetic foot which minimizes or eliminates “kickback” forces when the foot is utilized to walk over a door jamb or other raised profile object on a floor or on the ground, as well as reduce vibrations.

### SUMMARY OF THE INVENTION

An exemplary prosthetic foot can comprise a resilient bottom member having an anterior bottom end and a posterior bottom end, a resilient top member having an anterior top end and a posterior top end, wherein the anterior top end is connected to the anterior bottom end of the resilient bottom member, and wherein the resilient top member is positioned over the resilient bottom member and directed towards the posterior of the prosthetic foot, and an elastomeric bumper member comprising a tapered surface configured to contact the resilient bottom member and attached to an underside of the posterior top end of the resilient top member, wherein the bumper member is vertically oriented with respect to the prosthetic foot.

Furthermore, in another embodiment, a prosthetic foot can comprises a resilient bottom member having a first bottom end and a second bottom end, a resilient top member having a first top end and a second top end, wherein the first top end is connected to the first bottom end of the resilient bottom member, and wherein the resilient top member is positioned over the resilient bottom member and directed towards the back of the prosthetic foot, and a toe pad. The toe pad can comprise at least one spacer coupled to, and creating space between, the first bottom end of the bottom member and the first top end of the top member, and an adhesive bonding the first bottom end of the bottom member and the first top end of the top member, wherein the adhesive is commingled with the at least one spacer between the first bottom end and the first top end.

### BRIEF DESCRIPTION OF THE DRAWINGS

These and other features, aspects and advantages of the present invention will become better understood with reference to the following description, appending claims, and accompanying drawings where:

FIGS. 1A and 1B are perspective views illustrating a prosthetic foot constructed in accordance with various embodiments;

FIG. 2 is a rear view further illustrating the prosthetic foot of FIGS. 1A and 1B;

FIG. 3 is a side view further illustrating the prosthetic foot of FIGS. 1A and 1B;

FIGS. 4A and 4B are perspective views illustrating a prosthetic foot comprising a toe wrap;

FIGS. 5A-5C are side views illustrating various embodiments of a damper bar configuration;

FIG. 6 is a side view illustrating an exemplary prosthetic foot for use by an above-knee amputee; and

FIG. 7 is a side view illustrating an exemplary prosthetic foot for use by a below-knee amputee.

### DETAILED DESCRIPTION OF EXEMPLARY EMBODIMENTS

While exemplary embodiments are described herein in sufficient detail to enable those skilled in the art to practice the invention, it should be understood that other embodiments may be realized and that logical structural, material, and mechanical changes may be made without departing from the spirit and scope of the invention. Thus, the following descriptions are not intended as a limitation on the use or applicability of the invention, but instead, are provided merely to enable a full and complete description of exemplary embodiments.

Briefly, in accordance with exemplary embodiments, a prosthetic foot has improvements over a prior art prosthetic foot in that a more natural motion and response of the foot occurs during movement. In particular, the movement of the exemplary prosthetic foot replicates the natural flex of a foot and supplies continuous energy to a person when striding from heel to toe.

In the exemplary embodiment a prosthetic foot stores energy during the gait cycle and transfers the energy in order to “put a spring in your step.” The gait cycle, and specifically the stance phase, includes a heel strike phase, the mid-stance phase, and the toe-off phase. The heel strike phase begins when the heel of the foot touches the ground, and includes the loading response on the foot. The mid-stance phase is when the foot is flat on the ground and the body's center of gravity is over the foot. The toe-off phase is the finish of the stance phase and ends when the tip of the foot is the only portion in contact with the ground, and the load is entirely on the toe.

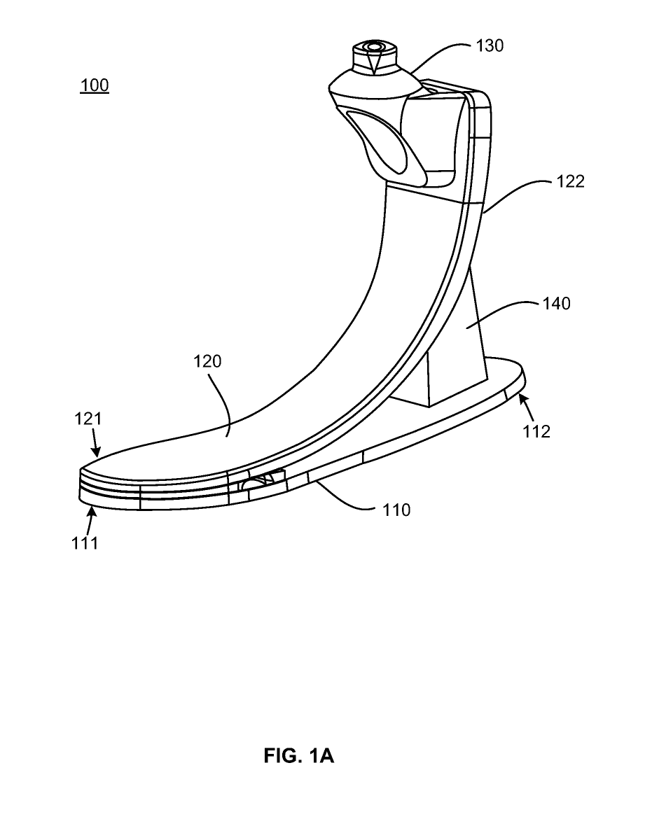

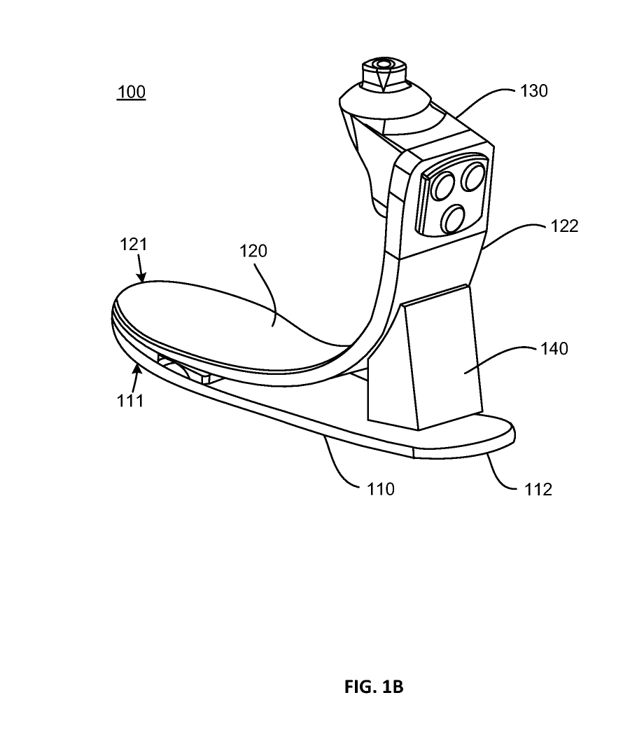

In accordance with various embodiments and with reference to FIGS. 1A and 1B, a prosthetic foot 100 comprises a resilient bottom member 110, a resilient top member 120, a connection point 130 attached to the top member 120 and configured for attachment to a user, and a bumper member 140. The resilient bottom member 110 may have an anterior bottom end 111 and a posterior bottom end 112. The resilient top member 120 may have an anterior top end 121 and a posterior top end 122. Further, the anterior top end 121 of the resilient top member 120 can be connected to the anterior bottom end 111 of the resilient bottom member 110, while the resilient top member 120 can be positioned over the resilient bottom member 120 and directed towards the posterior of the prosthetic foot 100.

Further, in various embodiments, prosthetic foot 100 also comprises an elastomeric bumper member 140 having a tapered surface configured to contact the resilient bottom member 110 and attached to an underside of the posterior top end 122 of the resilient top member 120. The bumper member 140 can be vertically oriented with respect to the prosthetic foot 100. The bumper member 140 can act as a heel shock for absorbing force on the downward strike during the user's stride.

In various embodiments, the bumper member 140 can be made from an elastomeric material. In one embodiment, the elastomeric material has about 80% or greater energy return. In another embodiment, the elastomeric material has about 90% or greater energy return. The bumper member 140 can be designed to behave similar to a non-linear spring, thereby allowing larger deflection of the posterior toe and 122 during the heel strike. The progressive “spring rate” may lead to a soft heel strike but does not deflect too far as the bumper member 140 compresses. One benefit of the bumper 140 is being relatively lightweight in comparison to a prosthetic feet with coiled springs.

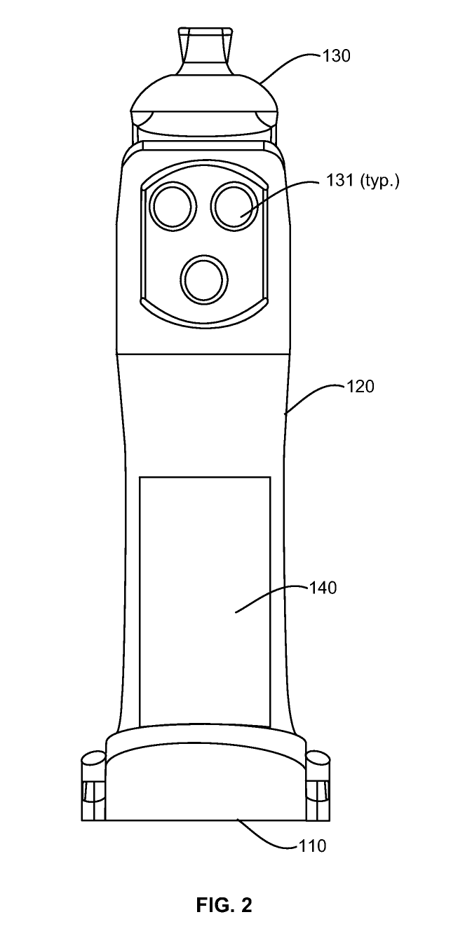

The bumper member 140 can be located posterior to vertical axis of the connection point 130. The bumper member 140 can be attached to the underside of the top member 120 in various manners. For example and with reference to FIG. 2, the bumper member 140 can be fixedly attached using adhesive or fasteners, such as screws. In another example, the bumper member 140 may be detachable using fasteners for replacement purposes. Moreover, in other embodiments, the bumper member 140 can be attached to various locations on the underside of the top member 120 or topside of the bottom member 110. In various embodiments, the prosthetic foot 100 in a static mode has a gap between the bumper member 140 and the bottom member 110. For example, a gap of about 1/10 inch may be present between the bumper member 140 and the bottom member 110. In other various methods, the bumper member 140 can be in contact with both the top member 120 and the bottom member 110 when the prosthetic foot 100 is in a static position. The lack of a gap results in the bumper member 140 being continuously compressed during the gait cycle, though the bumper member 140 is a compression member and not a tension member since the bumper member 140 is only attached to either the top member 120 or the bottom member 110.

The bumper member 140 can be in many shapes. In various embodiments, the detached portion of the bumper member 140 may have a conical, rectangular, or pyramid shape. The tapered surface of the bumper member 140 can terminate in an apex or hemispherical shape, and the apex can be configured to contact the bottom member 110 in response to deflection of the prosthetic foot 100. Moreover, in various embodiments, the bumper member 140 can terminate in multiple points. The tapered bumper member 140 facilitates a damping of vibration and sound generated during heel strike or release. Furthermore, in various embodiments the extruding portion of the bumper member 140 may be any shape that is non-flat surface. Further, a non-flat surface enhances lateral flexibility if the heel strike is not vertical.

The prosthetic foot 100 can be adjusted to accommodate a user in part by adjusting characteristics of the bumper member 140. For example, in various embodiments, the durometer of the bumper member 140 can be increased for users with more heel strike force, which may be caused by additional weight or dynamic activity. A heavier user may be better-suited using a bumper member with a large cross-sectional area compared to a lighter user using a bumper member with a small cross-sectional area.

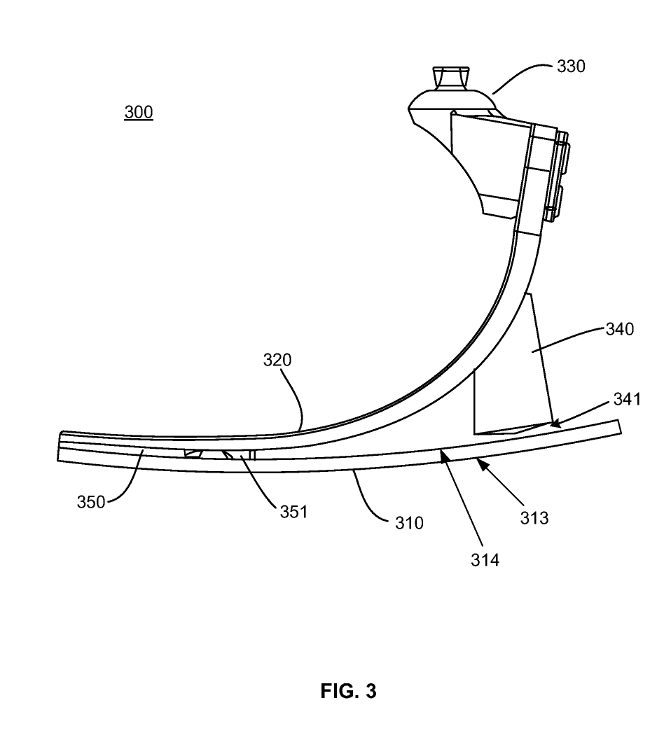

In accordance with various embodiments and with reference to FIG. 3, a prosthetic foot 300 comprises a resilient bottom member 310, a resilient top member 320, a connection point 330 attached to the top member and configured for attachment to a user, and a toe pad 350 coupled to the top surface of the bottom member 310 at a first bottom end and coupled to the bottom surface of the top member 320 at a first top end. Also, in various embodiments, prosthetic foot 300 may further comprise a bumper member 340. In various embodiments, the toe pad 350 comprises at least one spacer and an adhesive bonding the top surface of the bottom member 310 and the bottom surface of the top member 320. For example, the anterior quarter of the bottom member 310 can be adhesively connected to the top member 320. In various embodiments, adhesive can be used to connect 23-27% of the top surface area of the bottom member 310 to the top member 320. Further, in various embodiments, adhesive can be used to connect approximately ⅓ of the top surface area of the bottom member 310 to the top member 320.

In various embodiments, the toe pad 350 has approximately constant thickness. In other various embodiments, the toe pad 350 can have a thickness that tapers towards the front edge of the prosthetic foot 300. In other words, the toe pad 350 closer to the heel can be thicker than the toe pad 350 closer to the toe. Further, the adhesive bonding of the toe pad 350 can produce distributed stresses. In accordance with various embodiments, the adhesive can have a higher modulus of elasticity in contrast to the elastomer of the toe pad. Though other modulus values are contemplated, and various moduli may be used as well, a stiffer adhesive is preferred compared to a flexible adhesive.

The spacer of the toe pad 350 creates a space between the top surface of the bottom member 310 and the bottom surface of the top member 320. The adhesive can be commingled with the spacer between the top surface of the bottom member 310 and the toe pad 350 and also between the bottom surface of the top member 320 and the toe pad 350. In various embodiments, the space created by the spacer can be non-compressed space for the placement of the adhesive. In other words, the spacer can create a void between the top member 320 and the bottom member 310 and the void can be filled with the adhesive for bonding. The inclusion of the toe pad 350 may reduce the stress applied to the adhesive bond during the gait cycle. In various embodiments, the spacer can be elastomeric stand-offs, such as dots, ribs, or other patterns to create the desired spacing. Moreover, in various embodiments, the spacer is a single piece of connected stand-offs. The single piece spacer facilitates easier alignment during the manufacturing process and can provide a more uniform stand-off pattern compared to multiple stand-off spacers.

The toe pad 350 can also comprise an adhesive composite with spacers. In various embodiments of the prosthetic foot 300, the spacer is an aggregate material combined with the adhesive to form the adhesive composite. In various embodiments, the adhesive composite includes adhesive and microspheres. The microspheres can create the spacing between the top and bottom members 320, 310.

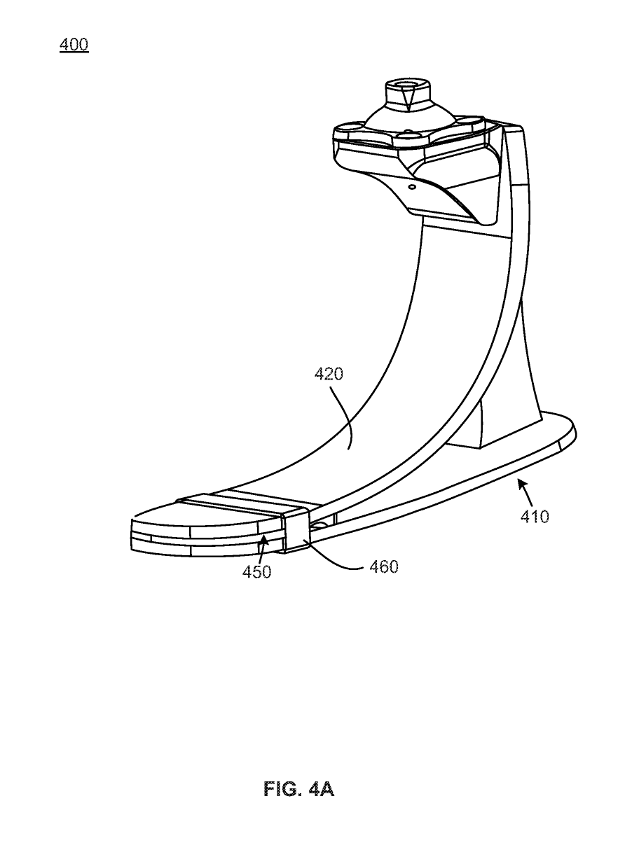

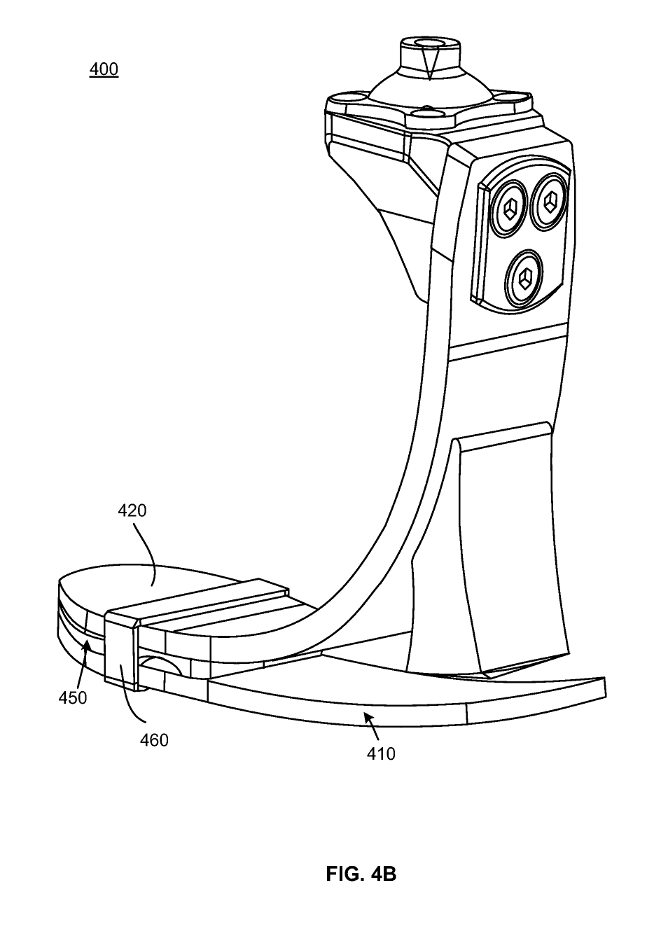

Additionally, in various embodiments and with reference to FIGS. 4A and 4B, a prosthetic foot 400 can comprise a bottom member 410, a top member 420, a toe pad 450, and a toe wrap 460 bonded around the top and bottom of the bonded bottom and top members 410, 420. The toe wrap 460 can be made out of a fiber material. The toe wrap material can also be a fiber weave with an elastomeric material. For example, the toe wrap can be a Kevlar or nylon material belt that is approximately less than a 1/10^{th} of an inch in thickness. The toe wrap 460 can be configured to provide a secondary hold in case the adhesive bond of the toe pad 450 between the top and bottom members breaks. Also, the toe wrap 460 can strengthen the attachment between the bottom and top members 410, 420 during tension.

Moreover, in various embodiments and with renewed reference to FIG. 3, the prosthetic foot 300 can further comprise a damper bar 351 configured to attach to an underside of the resilient top member 320 and contact the resilient bottom member 310. The damper bar 351 can be configured to arrest the upward motion of bottom member 310 after toe off and also arrest the rotational energy during the gait cycle. The arrested motion creates a slower velocity and less motion at the point of contact of the damper bar 351. Without the damper bar, the bottom member 310 may slap against the bumper member 340 during the stride, resulting in vibration traveling up the leg of the user.

In various embodiments, the damper bar 351 can be located near the posterior edge of the toe pad 350. As an example, the damper bar 351 can be spaced ½ inch away from the posterior edge of the toe pad 350. In another example, the damper bar 351 can be located in the anterior portion of the bottom member 310. Further, the damper bar 351 can be approximately a ½ inch long, with the length measured from anterior to posterior of the bottom member 310. In various embodiments, the width of the damper bar 351 can be as wide as the attached top member 320. However, the damper bar 351 may also be less than the full width of the attached top member 320. Furthermore, in various embodiments, the contacting surface of the damper bar 351 can be flat. In alternative embodiments, the contacting surface of the damper bar 351 can be tapered to an apex. The contacting surface can be configured to reduce vibration and sounds caused from the contact of the non-connected bottom member 310 with the damper bar 351 during the gait cycle. Furthermore, in various embodiments, the contacting surface of the damper bar 351 can be various shapes other than flat, such as a preloaded taper.

In various embodiments, the damper bar 351 is connected to the toe pad 350, or is formed as part of the toe pad 350. One advantage of having the toe pad 350 and damper bar 351 as a single piece is for easier alignment during manufacturing of the prosthetic foot 300.

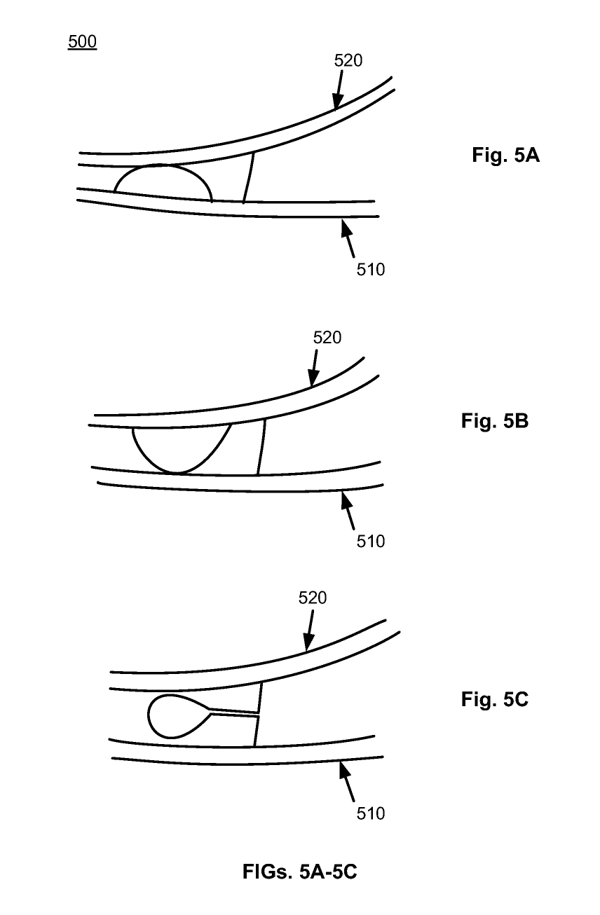

The damper bar 351 can be minimally load-bearing, whereas the bumper member 340 can be the primary load-bearing component. In various embodiments, the bumper member 340 can be located about four to five times farther back from the fulcrum point of the toe pad 350 in comparison to the damper bar 351. Furthermore, in various embodiments and with reference to FIGS. 5A-5C, a damper bar can be attached to the prosthetic foot in various configurations. For example, FIG. 5A illustrates a damper bar 551 attached to a top member 520, whereas FIG. 5B illustrates a damper bar 551 attached to a bottom member 510. In another example, FIG. 5C illustrates a damper bar 551 attached to both the bottom member 510 and the top member 520, where the damper bar 551 is divided such that the top and bottom member may separate and still arrest motion of the prosthetic foot.

Moreover and with renewed reference to FIGS. 1A and 1B, the top member 120, bottom member 110, and bumper member 140 transfer energy between themselves in a natural, true foot manner. The loading response during the heel strike phase compresses bumper member 140 and top member 120, which in turn passes energy into, and causes a deflection of, a rear portion of bottom member 110. Energy is transferred towards the front of prosthetic foot 100 during the mid-stance phase. Furthermore, an upward deflection of at least one of bottom member 110 and top member 120 stores energy during the transition from the mid-stance phase to the toe-off phase of the gait cycle. In an exemplary embodiment, about 90% or more of the heel strike loading energy is stored and transferred to top member 120 for assisting the toe-off phase. In another exemplary embodiment, about 95% or more of the heel strike loading energy is stored and transferred to top member 120 for assisting the toe-off phase. In yet another exemplary embodiment, about 98% or more of the heel strike loading energy is stored and transferred to top member 120 for assisting the toe-off phase. Prosthetic foot 100 may be designed to release the stored energy during the toe-off phase and assist in propelling the user in a forward direction.

In an exemplary embodiment and with renewed reference to FIG. 3, resilient bottom member 310 includes a bottom surface 313 and an upper surface 314. Resilient bumper member 340 includes a contact surface 341. When prosthetic foot 300 is compressed, resilient top member 320 and bumper member 340 are compressed and displaced downwardly toward resilient bottom member 310.

With respect to the walking motion, the prosthetic foot is configured to increase the surface-to-foot contact through the gait cycle. The increased surface contact allows for a smoother gait cycle, and increases stability in comparison to the typical prior art prosthetics. In exemplary embodiments, the underside of bottom member has different contours that provide increased surface contact for different types of uses.

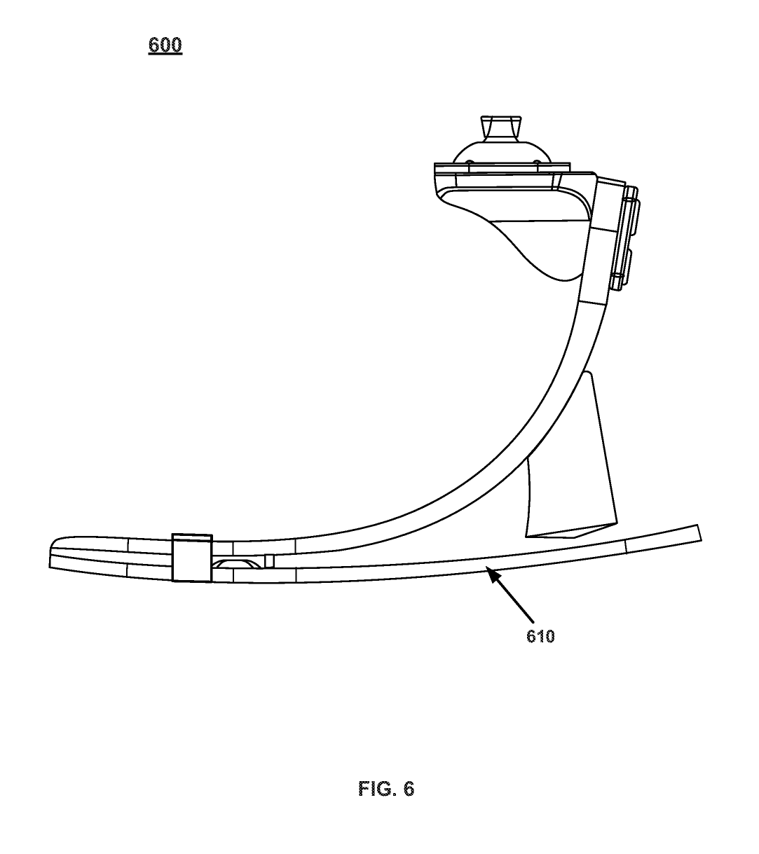

The bottom member of the prosthetic foot can have various shapes depending on desired use. The desired use may include prosthetic feet for above-knee amputees or prosthetic feet for below-knee amputees. In various embodiments and with reference to FIG. 6, a prosthetic foot 600 for above-knee amputees comprises a bottom member 610 having a curved bottom with no inflection point. In various embodiments, the bottom member 610 has a constant arc due to single radius forming the partial curve of the bottom member. In other various embodiments, the curve of the bottom member 610 can be designed as a spline of variable radii. The curve of bottom member 610 in above-knee prosthetic foot 600 facilitates keeping an artificial knee stable because the forces substantially restrict the knee from bending. The curved bottom member 610 enables a rocking motion even if the artificial knee is hyper-extended.

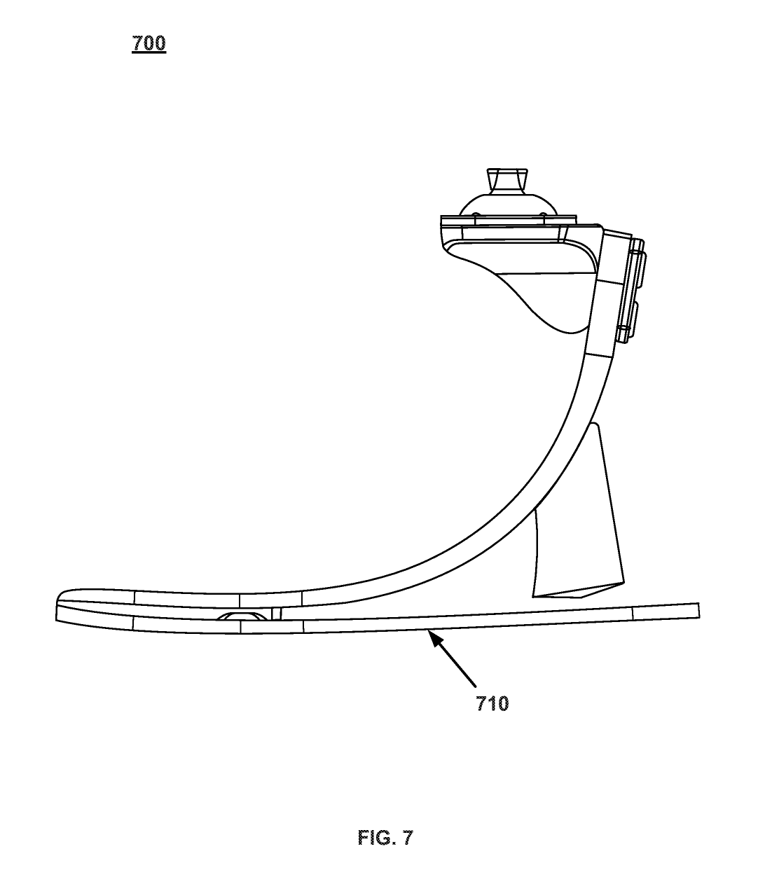

Similarly, in various embodiments and with reference to FIG. 7, a prosthetic foot 700 for below-knee amputees comprises a bottom member 710 having a partially curved portion in the anterior of the bottom member 710 and a substantially linear portion in the posterior portion of the bottom member 710. Similar to above-knee prosthetic foot 600, the anterior portion of bottom member 710 can have a constant arc due to single radius forming the partial curve. In various embodiments, the anterior portion of bottom member 710 can have a curve designed as a spline of variable radii. In accordance with various embodiments, the posterior portion of bottom member 710 can be substantially straight and tangent to the anterior portion such that bottom member 710 does not have an inflection point. A straight posterior portion and a curved anterior portion of bottom member 710 in below-knee prosthetic foot 700 facilitates rotation of the tibia progressing the natural rotation of the knee forward and preventing hyper-extension of the knee.

In accordance with an exemplary embodiment, resilient members 110, 120 are made of glass fiber composite. The glass fiber composite may be a glass reinforced unidirectional fiber composite. In one embodiment, the fiber composite material is made of multiple layers of unidirectional fibers and resin to produce a strong and flexible material. The fibers may be glass fibers or carbon fibers. Specifically, layers of fiber are impregnated with the resin, and a glass reinforcement layer can be positioned between at least two fiber weave layers. Typically, several layers of the unidirectional fibers or tape are layered together to achieve the desired strength and flexibility. Further, in various embodiments the layers of unidirectional fibers or tape can be oriented at various angles.

In the following description and/or claims, the terms coupled and/or connected, along with their derivatives, may be used. In particular embodiments, connected may be used to indicate that two or more elements are in direct physical contact with each other. Coupled may mean that two or more elements are in direct physical contact. However, coupled may also mean that two or more elements may not be in direct contact with each other, but yet may still cooperate and/or interact with each other. Furthermore, the term “and/or” may mean “and”, it may mean “or”, it may mean “exclusive-or”, it may mean “one”, it may mean “some, but not all”, it may mean “neither”, and/or it may mean “both”, although the scope of claimed subject matter is not limited in this respect.

It should be appreciated that the particular implementations shown and described herein are illustrative of various embodiments including its best mode, and are not intended to limit the scope of the present disclosure in any way. While the principles of the disclosure have been shown in embodiments, many modifications of structure, arrangements, proportions, the elements, materials and components, used in practice, which are particularly adapted for a specific environment and operating requirements without departing from the principles and scope of this disclosure. These and other changes or modifications are intended to be included within the scope of the present disclosure and may be expressed in the following claims.

1. A prosthetic foot, comprising:
a resilient bottom member comprising a front end, a middle, and a rear end, and having no inflection point, wherein a radius of curvature from the front end to the middle of the resilient bottom member is above the bottom member and the resilient member is substantially straight from the middle to the rear end;
a resilient top member comprising a front end and a rear end, wherein the front end is connected to the front end of the resilient bottom member, and wherein the resilient top member is positioned over the resilient bottom member; and
a bumper member attached to one of an underside of the rear end of the resilient top member and an upper side of the rear end of the resilient bottom member and disengaged in the unloaded state from the other of the underside of the rear end of the resilient top member and the upper side of the rear end of the resilient bottom member.

2. The prosthetic foot of claim 1, wherein the radius of curvature upon loading from the front end to the middle of the resilient bottom member is above the bottom member.

3. The prosthetic foot of claim 1, wherein the resilient member is substantially straight from the middle to the rear end in the unloaded state;

4. The prosthetic foot of claim 1, further comprising a toe piece coupling the front end of the resilient bottom member to the front end of the resilient top member.

5. The prosthetic foot of claim 4, wherein the toe piece comprises at least one spacer coupled to, and creating a space between, the front end of the resilient bottom member and the front end of the resilient top member.

6. The prosthetic foot of claim 5, further comprising an adhesive bonding the front end of the resilient bottom member to the front end of the resilient top member or an adhesive commingled with the at least one spacer.

7. The prosthetic foot of claim 4, wherein the toe piece comprises an elastomer material.

8. The prosthetic foot of claim 1, wherein the resilient top member and the resilient bottom member store energy during deflection.

9. The prosthetic foot of claim 1, wherein the bumper member is coupled to an underside of the rear end of the resilient top member and configured to contact and disengage the resilient bottom member upon compression and decompression, respectively, of the foot.

10. The prosthetic foot of claim 1, wherein the bumper member is an elastomeric material.

11. The prosthetic foot of claim 1, further comprising a damper bar attached to one of an underside of the resilient top member or a topside of the resilient bottom member and configured to contact the resilient bottom member upon compression of the foot.

12. The prosthetic foot of claim 11, wherein the damper bar is integrated into the toe piece.

13. The prosthetic foot of claim 11, wherein the damper bar is located adjacent a posterior portion of the toe piece.

14. The prosthetic foot of claim 11, wherein the damper bar comprises a vibration dampener configured to reduce at least one of vibration and sound caused by contact of the bottom member with the damper bar.

15. A prosthetic foot, comprising:
a resilient bottom member comprising a front end, a middle, and a rear end, wherein the resilient bottom member comprises a substantially straight rear portion in an unloaded state;
a resilient top member comprising a front end and a rear end, wherein the front end is coupled to the front end of the resilient bottom member, and wherein the resilient top member is positioned over the resilient bottom member; and
a bumper member;
attached to only one of an underside of the rear end of the resilient top member and an upper side of the rear end of the resilient bottom member; and
disengaged in an unloaded state from the other of the underside of the rear end of the resilient top member and the upper side of the rear end of the resilient bottom member.

16. The prosthetic foot of claim 15, wherein the resilient bottom member comprises a partially curved front portion.

17. The prosthetic foot of claim 16, wherein the resilient bottom member comprises no inflection point.

18. The prosthetic foot of claim 16, wherein a radius of curvature from the front end to the middle of the resilient bottom member is above the bottom member.

19. The prosthetic foot of claim 15, wherein the bumper member further comprises a tapered surface configured to contact and to disengage from one of an underside of the rear end of the resilient top member and an upper side of the rear end of the resilient bottom member in response to deflection of the prosthetic foot.

20. A prosthetic foot, comprising:
a resilient bottom member, comprising:
a front end;
a middle;
a rear end;
an at least partially curved portion from the front end to the middle in an unloaded state; and
a substantially straight portion from the middle to the rear end in the unloaded state;
a resilient top member comprising an outer arced surfaced, a front end, and a rear end, wherein the front end of the resilient top member is connected to the front end of the resilient bottom member, and wherein the resilient top member is positioned over the resilient bottom member; and
an elastomeric bumper member attached to an underside of the rear end of the outer arced surface of the resilient top member and vertically oriented with respect to the prosthetic foot and configured to:
contact the resilient bottom member in response to a compressive force applied to the foot; and
disengage from the resilient bottom member upon removal of the compressive force.

21. The prosthetic foot of claim 20, wherein the resilient bottom member comprises no inflection point.

22. The prosthetic foot of claim 20, wherein a radius of curvature from the front end to the middle of the resilient bottom member is above the bottom member.

23. The prosthetic foot of claim 20, wherein the bumper member comprises a tapered surface.

24. The prosthetic foot of claim 23, wherein the tapered surface of the bumper member terminates in an apex, and wherein the apex is configured to contact the bottom member in response to deflection of the prosthetic foot.
